(19) United States
(12) Patent Application Publication
(10) Pub. No.: US 2012/0108672 A1
(43) Pub. Date: May 3, 2012

(54) OPHTHALMIC COMPOSITION AND METHOD FOR PREVENTION OF CLOUDINESS/PRECIPITATION

(76) Inventors: Hazuki Tsutsui, Tokyo (JP); Miyuki Miyake, Tokyo (JP); Akito Odaka, Tokyo (JP); Chieko Inoue, Tokyo (JP); Nobuhito Tabuchi, Tokyo (JP); Manabu Hattori, Tokyo (JP)

(21) Appl. No.: 13/380,637

(22) PCT Filed: Jun. 23, 2010
(86) PCT No.: PCT/JP2010/060633

§ 371 Date: Dec. 23, 2011

(30) Foreign Application Priority Data
Jun. 25, 2009 (JP) 2009-150874
Jun. 30, 2009 (JP) 2009-155410

(51) Int. Cl. A61K 31/07; A61P 27/02

(52) U.S. Cl. 514/725

(57) ABSTRACT

An ophthalmic composition characterized by comprising (A) vitamin A, (B) a polyoxyethylene polyoxypropylene glycol, and at least one component selected from (C) trometamol, (D) polyhydric alcohols, (E) a sugar, (F) phosphoric acid and salt thereof and (G) monovalent neutral salts.

### TECHNICAL FIELD

This invention relates to an ophthalmic composition containing vitamin A and more particularly, to an ophthalmic composition that ensures good storage stability of vitamin A and does not undergo cloudiness/precipitation as will be caused by freeze and thaw thereof and is thus stable in appearance and also to a method for preventing cloudiness/precipitation ascribed to the freeze and thaw of this composition. Moreover, the invention relates to a dry eye remedy having a corneal/conjunctival disorder treatment effect and containing vitamin A.

### BACKGROUND ART

Vitamin A has drawn attention as an ingredient effective for preventing or treating keratoses of the cornea/conjunctiva and the skin mucosa. In recent years, it has been reported that vitamin A has an effect on dry eye symptoms such as corneal and conjunctival xerosis. However, lipophilic vitamin A is very sensitive to air, light, heat, acid, metal ion and the like and especially, it is very unstable in aqueous solutions. Thus, it has been made difficult to stably formulate it in ophthalmic compositions such as eye drops.

Stabilizing techniques of such unstable vitamin A have been hitherto proposed including a method of stabilization with nonionic surfactants such as polyoxyethylene hardened castor oil or the like (see Patent Documents 1, 2: JP-A H05-331056 and JP-A H06-40907), a method of stabilization with vitamin E's that are a hydrophobic antioxidant (see Patent Document 3: JP-A H06-247853), and a technique of stabilization from the aspects of a container and package (see Patent Document 4: JP-A 2003-113078) and a stabilization technique based on its preparation by high-energy emulsification (see Patent Document 5: JP-A 2002-332225).

Dry eye means a state of the cornea and conjunctiva on the surface of the eyeball, which undergo a disorder associated with the qualitative or quantitative abnormality of tears. The tear fluid is constituted of three layers including an oil layer, an aqueous layer and a mucin layer and when the qualitative and quantitative balance of this three-layered structure is destroyed, the tear fluid becomes unstable, so that the cornea is damaged, thereby bringing about eye dryness. For treating dry eye symptoms, it is important to restore the three-layered structure of the oil layer, aqueous layer and mucin layer of the tear fluid and subject to corneal disorder treatment.

It is known that vitamin A is essential for the proliferation and differentiation of epithelial cells, and it has been reported that vitamin A has the action of promoting mucin production (see, for example, Non-Patent Document 1: Kubo, Y., J Jpn Ophthalmol Sci. 103, 580-583, 1999) and also the action of healing a corneal wound (see, for example, Non-Patent Document 2: Ubels, J. L., Curr. Eye Res. 4, 1049-1057, 1985). As set out above, vitamin A has been expected as a drug that shows the effect on “the restoration of the mucin layer of tear fluid” and “the treatment of corneal disorder” and is thus useful for dry eye treatment. Gathering the foregoing, there has been demanded a dry eye remedy that exhibits a high dry eye treatment effect and contains vitamin A.

### PRIOR ART DOCUMENTS

### Patent Documents

Patent Document 1: JP-A H05-331056
Patent Document 2: JP-A H06-40907
Patent Document 3: JP-A H06-247853
Patent Document 4: JP-A 2003-113078
Patent Document 5: JP-A 2002-332225
Patent Document 6: JP-A 2001-322936

### Non-Patent Documents

Non-Patent Document 1: Kubo, Y., J Jpn Ophthalmol Sci. 103, 580-583, 1999)
Non-Patent Document 2: Ubels, J. L., Curr. Eye Res. 4, 1049-1057, 1985)

### SUMMARY OF THE INVENTION

### Problems to be Solved by the Invention

We have made studies so as to realize a higher degree of stabilization of vitamin A and particularly, to obtain a stable ophthalmic composition in such a concentration range of vitamin A as will be difficult in securing stability, under which polyoxyethylene polyoxypropylene glycol has been selected as an excellent stabilizing ingredient. However, with the preparations formulated with polyoxyethylene polyoxypropylene glycol, it has been clarified that problems are involved in that when they are stored at low temperature, especially, under frozen conditions, cloudiness or white precipitation occurs upon thawing, and when freeze and thaw are repeated, an appearance becomes further worsened. Generally, for the storage of eye drops, it is considered to store them at room temperature or in refrigerator. In a more extreme case, it is assumed that eye drops are allowed to stand in a low temperature condition such as in refrigerator or to stand in a cold district in winter, thereby causing them to be frozen. Accordingly, there has been demanded an improvement in storage stability upon storage in a cold place or upon freezing and thawing.

The invention has been made under such circumstances as set out above and has for its object the provision of an ophthalmic composition containing vitamin A and polyoxyethylene polyoxypropylene glycol, which is excellent in storage stability of vitamin A and does not cause cloudiness/precipitation upon freezing and thawing along with its appearance being kept stably and also of a method for preventing cloudiness/precipitation of the composition as will be caused by freeze and thaw of the composition.

The invention also has as its object the provision of a dry eye remedy wherein the corneal and conjunctival damage treatment effect of vitamin A is improved.

### Means for Solving the Problems

We have made intensive studies in order to achieve the above objects and, as a result, found that when an ophthalmic composition including (A) vitamin A and (B) polyoxyethylene polyoxypropylene glycol is further formulated with one or two or more, preferably two or more, of ingredients selected from (C) to (G) ingredients: (C) trometamol, (D) a polyhydric alcohol, (E) a sugar, (F) phosphoric acid and its salt, and (G) a monovalent neutral salt, the storage stability of vitamin A becomes excellent and cloudiness/precipitation in the course of freeze and thaw can be prevented, thereby arriving at completion of the invention.

Although the detailed mechanism as to how to prevent the cloudiness/precipitation in the course of freeze and thaw is not known, polyoxyethylene polyoxypropylene glycol has a narrow L1 micellar region of its aqueous solution relative to the concentration thereof and is thus liable to be converted to a viscous gel state only by a slight degree of condensation. In contrast, with nonionic surfactants such as polyoxyethylene hardened castor oil, polyoxyethylene sorbitan fatty acid esters and the like, the L1 micellar region is extended toward to a high concentration side, resulting in the unlikelihood of receiving a condensation effect. More particularly, the cloudiness/precipitation in the course of freeze and thaw is a problem involved inherently in polyoxyethylene polyoxypropylene glycol.

When hydrated water at the ethylene oxide chains of polyoxyethylene polyoxypropylene glycol is frozen, the free volume of the ethylene oxide chains decreases, so that the fill form of the molecules of the polyoxyethylene polyoxypropylene glycol is changed to a form that is likely to take an associated state whose curvature is smaller than spherical micelles have. It may be considered that micellar cores mutually agglutinate based on the orientation/eccentric location of vitamin A already frozen, thereby causing precipitation in the form of a clouded matter.

On the other hand, it may be considered as well that cloudiness/precipitation in the course of freeze and thaw can be prevented by adding the above-indicated cloudiness/precipitation preventive ingredients so as to prevent bulk water from being frozen and to allow the ingredients to be infiltrated into the ethylene oxide chains of the micelles thereby disturbing the orientation of the ethylene oxide chains to prevent the ethylene oxide chains from being frozen and thus stabilize the associated state of the micelles.

Accordingly, the invention provides the following ophthalmic composition and method for preventing cloudiness/precipitation of the composition ascribed to the freeze and thaw thereof.

[1]. An ophthalmic composition, characterized by comprising (A) vitamin A, (B) polyoxyethylene polyoxypropylene glycol, and one or two or more selected from the group consisting of (C) trometamol, (D) a polyhydric alcohol, (E) a sugar, (F) phosphoric acid and its salt, and (G) a monovalent neutral salt.
[2]. The ophthalmic composition of [1], wherein two or more selected from the ingredients (C) to (G) are contained.
[3]. The ophthalmic composition of [1] or [2], wherein the ingredient (D) is glycerine, the ingredient (E) is xylitol, sorbitol, mannitol or trehalose, the ingredient (F) is sodium dihydrogen phosphate, and the ingredient (G) is sodium chloride.
[4]. The ophthalmic composition of any one of [1] to [3], wherein a total amount of the ingredients (C) to (G) is 0.001 to 5 W/V %.
[5]. The ophthalmic composition of any one of [1] to [4], wherein an amount of the ingredient (B) is not larger than 5 W/V %.
[6]. The ophthalmic composition of any one of [1] to [5], wherein the ingredient (A) is one or two or more selected from the group consisting of retinol palmitate, retinol acetate and retinoic acid.
[7]. The ophthalmic composition of any one of [1] to [6], wherein an amount of the ingredient (A) is 50,000 to 500,000 units/100 ml.
[8]. The ophthalmic composition of any one of [1] to [7], wherein an amount of a cationic surfactant and a hydrophobic antiseptic are not larger than 0.004 W/V %, respectively.
[9]. The ophthalmic composition of any one of [1] to [7], wherein no antiseptic is formulated.
[10]. The ophthalmic composition of any one of [1] to [9], wherein said composition is the ophthalmic composition for contact lens.
[11]. The ophthalmic composition of any one of [7] to [10], wherein said composition is dry eye remedy.
[12]. A method for preventing cloudiness/precipitation in the course of freezing and thawing comprising;

formulating one or two or more selected from the group consisting of (C) trometamol, (D) a polyhydric alcohol, (E) a sugar, (F) phosphoric acid and its salt, and (G) a monovalent neutral salt in an ophthalmic composition containing (A) vitamin A and (B) polyoxyethylene polyoxypropylene glycol.

### Advantageous Effect of the Invention

According to the invention, there can be provided an ophthalmic composition that is stably formulated with vitamin A and has a stable appearance without causing cloudiness/precipitation even when frozen and thawed and a method for preventing cloudiness/precipitation in the course of freeze and thaw of the composition.

### EMBODIMENT FOR CARRYING OUT THE INVENTION

The ophthalmic composition of the invention includes (A) vitamin A, (B) polyoxyethylene polyoxypropylene glycol, and one or two or more selected from the group consisting of (C) trometamol, (D) a polyhydric alcohol, (E) a sugar, (F) phosphoric acid and its salt, and (G) a monovalent neutral salt.

### (A) Vitamin A

Vitamin A includes vitamin A itself, vitamin A-containing mixtures such as vitamin A oil, vitamin A derivatives such as vitamin A fatty acid esters, and the like. More particularly, mention is made of retinol palmitate, retinol acetate, retinol, retinoic acid, retinoide and the like. Of these, retinol palmitate, retinol acetate and retinoic acid are preferred. Retinol palmitate is commercially sold usually as having 1,000,000 to 1,800,000 international units (hereinafter abbreviated as I.U.), for which specific mention is made of “retinol palmitate” (1,700,000 I.U./g), made by DSM Nutrition Japan K.K.).

The ingredients (A) may be used singly or in appropriate combination of two or more. The amount is preferably 50,000 to 500,000 units/100 ml, more preferably 50,000 to 300,000 units/100 ml, and further preferably 100,000 to 200,000 units/100 ml in the total amount of the ophthalmic composition. When expressed by W (weight)/V (volume) % (g/100 ml), the amount is preferably 0.03 to 0.3 W/V %, more preferably 0.03 to 0.18 W/V % and further preferably 0.06 to 0.12 W/V % although depending on the units of vitamin A being formulated. Vitamin A has a corneal/conjunctival damage treatment effect and an amelioration effect on dry eye, tired eye and bleary eye conditions. If the amount is less than 50,000 units/100 ml, there is concern that the corneal/conjunctival damage treatment effect becomes unsatisfactory. Over 500,000 units/100 ml, there is concern that problems of side effects may develop.

### (B) Polyoxyethylene Polyoxypropylene Glycol

Polyoxyethylene polyoxypropylene glycol is not particularly limited in type and those described in Japanese Pharmaceutical Excipients (JPE) may be used. The average degree of polymerization of ethylene oxide is preferably 4 to 200, more preferably 20 to 200 and the average degree of polymerization of propylene oxide is preferably 5 to 100, more preferably 20 to 70, and either a block copolymer or a random polymer may be used.

In particular, examples of polyoxyethylene polyoxypropylene glycol include polyoxyethylene (200) polyoxypropylene (70) glycols such as Lutrol F127 (made by BASF), Uniloob 70DP-950B (made by NFO Corporation) and the like, polyoxyethylene (196) polyoxypropylene (67) glycol (Pluronic F127, also known as Poloxmer 407), polyoxyethylene (120) polyoxypropylene (40) glycol (Pluronic F-87), polyoxyethylene (160) polyoxypropylene (30) glycol ((Pluronic F-68, otherwise known as Poloxmer 188) such as Plonon #188 (made by NFO corporation) and the like, polyoxyethylene (42) polyoxypropylene (67) glycol (Pluronic P123, otherwise known as Poloxamer 403), polyoxyethylene (54) polyoxypropylene (39) glycol (Pluronic P85) such as Plonon #235P (made by NFO Corporation) and the like, polyoxyethylene (20) polyoxypropylene (20) glycol (Pluronic L-44), Tetronic and the like. Of these, polyoxyethylene (200) polyoxypropylene (70) glycol, polyoxyethylene (160) polyoxypropylene (30) glycol, and polyoxyethylene (54) polyoxypropylene (39) glycol are preferred.

The ingredients (B) may be used singly or in appropriate combination of two or more. The amount in the ophthalmic composition is preferably not larger than 5 W/V %, more preferably 0.4 to 5 W/V % from the standpoints of the storage stability of vitamin A and also of the effects on the corneal and conjunctival damage treatment and dry eye treatment. If the amount is less than 0.4 W/V %, there is concern that a difficulty is involved in solubilizing vitamin A. The cloudiness/precipitation in the course of freeze and thaw is more unlikely to occur if the amount of the ingredient (B) is smaller. In this view, the content of the ingredient (B) is preferably not greater than 5 W/V %.

### (C) to (G) Cloudiness/Precipitation Preventing Ingredients (C) Trometamol

The amount of trometamol (C) is preferably at 0.001 to 5 W/V %, more preferably at 0.01 to 3 W/V % and further preferably at 0.1 to 2 W/V % in the ophthalmic composition. If trometamol is formulated at not less than 0.001 W/V %, better cloudiness/precipitation prevention can be attained. A larger amount of trometamol leads to a better cloudiness/precipitation preventing effect. In this connection, however, over 5 W/V %, the osmotic pressure would rise too much, with concern that stimulation is felt.

### (D) Polyhydric Alcohol

Examples of polyhydric alcohol include glycerine, propylene glycol, butylene glycol, polyethylene glycol and the like. Of these, glycerine and propylene glycol are preferred and glycerine is more preferred.

The amount of a polyhydric alcohol is, for example, preferably 0.001 to 5 W/V %, more preferably 0.005 to 3 W/V % and further preferably 0.01 to 2 W/V % in the ophthalmic composition. If the amount is less than 0.001 W/V %, an antifreezing effect is so weak that cloudiness/precipitation would not be prevented. Over 5 W/V %, there may be the case where an osmotic pressure would rise too much.

### (E) Sugars

Examples of sugar include glucose, cyclodextrin, xylitol, sorbitol, mannitol, trehalose and the like. These may be any of d isomer, l isomer or dl isomer. Of these, xylitol, sorbitol, mannitol and trehalose are preferred, sorbitol, mannitol and trehalose are more preferred, and mannitol and trehalose are much more preferred.

The amount of sugars is, for example, preferably 0.001 to 5 W/V %, more preferably 0.005 to 3 W/V %, further preferably 0.01 to 2 W/V % and most preferably 0.05 to 1 W/V % in the ophthalmic composition. If the amount is less than 0.001 W/V %, an antifreezing effect is so small that there would be the case where cloudiness/precipitation are not prevented. Over 5 W/V %, there would be the case where an osmotic pressure rises too much.

### (F) Phosphoric Acid and Salts Thereof.

Examples of phosphoric acid and salts thereof include phosphoric acid, monosodium phosphate, sodium dihydrogen phosphate, sodium hydrogen phosphate, trisodium phosphate, disodium hydrogen phosphate, potassium dihydrogen phosphate, potassium dihydrogen phosphate and the like. Of these, monosodium phosphate, sodium dihydrogen phosphate, sodium hydrogen phosphate, trisodium phosphate and disodium hydrogen phosphate are preferred, sodium dihydrogen phosphate, sodium hydrogen phosphate and disodium hydrogen phosphate are more preferred, and disodium hydrogen phosphate is much more preferred. The amount of phosphoric acid and its salt is, for example, preferably 0.001 to 5 W.V %, more preferably 0.005 to 3 W/V %, further preferably 0.01 to 2 W/V % and most preferably at 0.05 to 1 W/V % in the ophthalmic composition. If the amount is less than 0.001 W/V %, an antifreezing effect is so poor that cloudiness/precipitation would not be prevented in some case. Over 5 W/V %, there would be the case where an osmotic pressure rises too much.

### (G) Monovalent Neutral Salt

Examples of monovalent neutral salt include sodium chloride, potassium chloride and the like. Of these, sodium chloride is preferred. The amount of the monovalent neutral salt in the ophthalmic composition is preferably 0.001 to 5 W/V %, more preferably 0.01 to 3 W/V %, further preferably at 0.1 to 2 W/V % and most preferably at 0.1 to 1 W/V %. If the amount is less than 0.001 W/V %, an antifreezing effect would be so poor cloudiness/precipitation would not be prevented in some case. Over 5 W/V %, there would be the case where an osmotic pressure would rise too much.

As a cloudiness/precipitation preventing ingredient, trometamol (C) is preferred. These cloudiness/precipitation preventing ingredients may be used singly or in appropriate combination of two or more. Two or more types of same ingredients may be used, e.g. two or more ingredients (D) may be used in combination. The use in combination of two or more ingredients is more preferred from the standpoint that a synergistic effect is obtainable with respect to the freeze-preventing effect on bulk water and the hydrated water of ethylene oxide chains. Of these ingredients, it is particularly preferred to use trometamol (C) and other types of ingredients in combination. It is preferred from the standpoint of the freeze-preventing effect on the hydrated water of ethylene oxide chains of polyoxyethylene polyoxypropylene glycol to use two or more of glycerine, trometamol and trehalose, especially, glycerine and trometamol. For instance, trometamol not only contributes to freeze prevention of bulk water, but also joins to the ethylene oxide chains of micelles, and glycerine infiltrates into the ethylene oxide chains, for which it is considered that the orientation of the ethylene oxide chains is disturbed, thereby enabling the ethylene oxide chains to be prevented from freezing. In view of an improved storage stability of vitamin A, it is preferred to formulate trometamol in the ophthalmic composition of the invention. Although not yet known, this mechanism may be considered in the following way, for example. Polyoxyethylene polyoxypropylene glycol is a nonionic surfactant having a polyoxyethylene (EO) chain and a polyoxypropylene (PO) chain. Vitamin A is wrapped with the EO chain kept outside and also with the PO chain kept inside, thereby forming a micelle. The coexistence of trometamol permits the —NH₂ group present in trometamol to be directly bound to the ether bond of the EO chain, resulting in the strong structure of the micelle. Moreover, trometamol binds to the EO chain located at the outside of the micelle so that the micelle structure is rendered strong to lower a degree of freedom, eventually leading to the lowering of molecular mobility of the PO chain inside the micelle. From the above, it is considered that trometamol contributes to the stabilization of the micelle formed from vitamin A and polyoxyethylene polyoxypropylene glycol and, as a consequence, contributes to the storage stability of vitamin A.

The total amount of the ingredients (C) to (G) is preferably 0.001 to 5 W/V % in the ophthalmic composition. Especially, in case where two types of ingredients are used in combination, the total amount in the ophthalmic composition is preferably 0.01 to 5 W/V %, more preferably 0.1 to 4 W/V %, further preferably at 0.5 to 3 W/V %, and most preferably at 1 to 3 W/V %. Moreover, if three or more types of ingredients are used in combination, the total amount is more preferably 0.01 to 5 W/V % and much more preferably at 0.1 to 4 W/V %.

The total of the ingredients (C) to (G) is preferably 0.02 to 200 parts by weight per unit part by weight of the ingredient (A).

Moreover, the total of the ingredients (C) to (G) is preferably 0.001 to 20 parts by weight per unit part by weight of the ingredients (A)+(B).

### Other Ingredients

The ophthalmic composition of the invention may be further formulated, aside from the above-stated ingredients, with a variety of ingredients formulated in ophthalmic compositions within ranges not impeding the effects of the invention. These ingredients include surfactants other than the ingredient (B), buffering agents, thickening agents, pH adjusters, antiseptics, tonicity agents, stabilizing agents, cooling agents, drugs, water and the like. These may be used singly or in combination of two or more and appropriate amounts may be formulated.

(i) Surfactants Other than Ingredient (B)

Examples of surfactant other than the ingredient (B) include nonionic surfactants such as polyoxyethylene hardened castor oil, polyoxyethylene sorbitan fatty acid esters and the like, and glycine-based amphoteric surfactants such as alkyldiaminoethylglycines. The amount of these surfactants in the ophthalmic composition is preferably at 0.0001 to 10 W/V %, more preferably at 0.005 to 5 W/V %. In this regard, however, these surfactants should be favorably used in a reduced amount form the viewpoint of the effects on corneal damage treatment and dry eye treatment and the amount thereof is preferably at less than 0.5 W/V %.

### (ii) Antiseptics

Although the antiseptic may be formulated within a range of not impeding the effects of the invention, no formulation of an antiseptic wherein an antiseptic is absent is preferred from the standpoint of eye irritation. Examples of antiseptic include benzalkonium chloride, benzethonium chloride, sorbic acid or a salt thereof, paraoxybenzoic acid esters (such as methylparaben, ethylparaben, propylparaben and the like), chlorhexidine gluconate, thimerosal, phenyl ethyl alcohol, alkyldiaminoethylglycine hydrochloride, polyhexanide hydrochloride, Polidronium hydrochloride and the like. The amount of the antiseptic relative to the total amount of the ophthalmic composition is, for example, at 0.00001 to 5 W/V %, preferably at 0.0001 to 3 W/V % and more preferably at 0.001 to 2 W/V %.

In this regard, however, it is known that cationic surfactants such as benzalkonium chloride, benzethonium chloride and the like and hydrophobic antiseptics such as parabens (methylparaben, ethylparaben, propylparaben and the like), chlorobutanol and the like have the action of blocking the corneal/conjunctival damage treatment effect. Accordingly, the amount of these ingredients is preferably at not larger than 0.004 W/V % and more preferably at not larger than 0.003 W/V % in the composition. No formulation wherein they are not contained is much more preferred. Although it is not known that these ingredients block the corneal/conjunctival damage treatment effect, polyoxyethylene polyoxypropylene glycol (B) forms micelles wrapping vitamin A with the EO chain kept outside and the PO chain kept inside. This micelle adsorbed on the cornea surface and vitamin A is absorbed inside the cornea. It is considered that cationic surfactants have high surface activity or hydrophobic antiseptics are high in hydrophobicity, so that the surface state of the micelle is changed thereby blocking the adsorption of vitamin A on the cornea. Eventually, the corneal damage treatment effect and dry eye improvement are inhibited. On the other hand, those having high hydrophilicity such as sorbic acid or its salt do not influence the inside state of micelle and do not block the absorption-expediting effect of vitamin A.

The antiseptic power in case where no antiseptic is formulated, it is better to formulate one or more, preferably two or more, of sodium edetate, boric acid and trometamol in combination. If there is used a unit dose container or a filtered container, no antiseptic formulation is possible.

(iii) Buffering Agent

Examples of buffering agent include boric acid or its salt (borax or the like), citric acid or its salt (sodium citrate or the like), tartaric acid or its salt (sodium tartarate or the like), gluconic acid or its salt (sodium gluconate or the like), acetic acid or its salt (sodium acetate or the like), and a variety of amino acids (epsilon-aminocaproic acid, potassium aspartate, aminoethylsulfonic acid, glutamic acid, sodium glutamate and the like). Trometamol of the ingredient (C) may also be used as a buffering agent and is preferred from the viewpoint of low irritation and the antiseptic effect of composition. Moreover, when boric acid or borax are used in combination, a high antiseptic effect can be obtained. It will be noted that in the practice of the invention, when boric acid, trometamol and citric acid or its salt are formulated, the stability of vitamin A is further improved. The amount of the buffering agent in the ophthalmic composition is preferably at 0.001 to 10 W/V %, more preferably at 0.01 to 5 W/V %.

### (iv) Thickening Agent

Examples of thickening agent include polyvinyl pyrrolidone, hydroxyethylcellulose, hydroxypropyl methylcellulose, methylcellulose, polyvinyl alcohol, sodium hyaluronate, sodium chondroitin sulfate, polyacrylic acid, carboxyvinyl polymer and the like. The formulation of these ingredients permits high retention, thereby leading to a more improved corneal/conjunctival damage treatment effect. The amount of the thickening agent, relative to the total amount of the ophthalmic composition is, for example, preferably at 0.001 to 10 W/V %, more preferably at 0.001 to 5 W/V % and much more preferably at 0.01 to 3 W/V %.

### (v) pH Adjuster

The use of an inorganic acid or inorganic alkali as a pH adjuster is preferred. For example, (diluted) hydrochloric acid can be mentioned as an inorganic acid. As an inorganic alkali, mention is made of sodium hydroxide, potassium hydroxide, sodium carbonate, sodium hydrogen carbonate and the like. Of these, hydrochloric acid and sodium hydroxide are preferred. The pH (20° C.) of the ophthalmic composition of the invention is preferably at 4.0 to 9.0, more preferably at 5.0 to 8.0 and much more preferably at 6.0 to 8.0. It will be noted that in the practice of the invention, the measurement of pH is carried out using a pH osmometer (HOSM-1, made by DKK-Toa Corporation). The amount of the pH adjuster, relative to the total amount of the ophthalmic composition is, for example, at 0.00001 to 10 W/V %, preferably at 0.0001 to 5 W/V % and much more preferably at 0.001 to 3 W/V %.

### (vi) Tonicity Agent

Examples of tonicity agent include calcium chloride, magnesium chloride and the like. The amount of the tonicity agent, relative to the total amount of the ophthalmic agent is, for example, at 0.001 to 5 W/V %, preferably at 0.01 to 3 W/V % and much more preferably at 0.1 to 2 W/V %.

(vii) Stabilizing Agent

Examples of stabilizing agent include sodium edetate, cyclodextrin, sulfites, dibutylhydroxytoluene and the like. It will be noted that in the invention, the formulation of the stabilizing agent leads to improved stability of vitamin A. The amount of the stabilizing agent, relative to the total amount of the ophthalmic agent, is, for example, at 0.001 to 5 W/V %, preferably at 0.01 to 3 W/V % and much more preferably at 0.1 to 2 W/V %.

(viii) Cooling Agent

Examples of cooling agent include menthol, camphor, borneol, geraniol, cineol, linalool and the like. The amount of the cooling agent in the ophthalmic composition as a total amount of compounds is preferably at 0.0001 to 5 W/V %, preferably at 0.001 to 2 W/V %, much more preferably at 0.005 to 1 W/V % and most preferably at 0.007 to 0.8 W/W.

### (ix) Drug (Pharmaceutically Effective Ingredient)

Examples of drug (pharmaceutically effective ingredient), there may be appropriately formulated, a decongestant (e.g. naphazoline hydrochloride, tetrahydrozoline hydrochloride, phenylephrine hydrochloride, epinephrine, ephedrine hydrochloride, dl-methylephedrine hydrochloride, tetrahydrozoline nitrate, naphazoline nitrate or the like); an antiphlogistic/astringent agent (e.g. neostigmine methylsulfate, E-aminocaproic acid, allantoin, berberine chloride, zinc sulfate, zinc lactate, lysozyme chloride, dipotassium glycyrrhizinate, ammonium glycyrrhizinate, glycyrrhetinic acid, methyl salicylate, tranexamic acid, azulene sodium sulfonate or the like); an antihistamine agent (e.g. iproheptine hydrochloride, diphenhydramine hydrochloride, diphenhydramine, isothipendyl hydrochloride, chlorpheniramine maleate or the like); anti-allergic agent (e.g. sodium cromoglicate, ketotifen fumarate, or the like), a water-soluble vitamin (activated vitamin B₂ vitamin B₆, vitamin B₁₂ or the like); an amino acid (e.g. potassium L-aspartate, magnesium L-aspartate, aminoethylsulfonic acid, sodium chondroitin sulfate or the like); a sulfa drug or bactericide (e.g. sulfur, isopropylmethylphenol, hinokitiol or the like); a regional anesthetic (e.g. lidocaine, lidocaine hydrochloride, procaine hydrochloride, dibucaine hydrochloride or the like); and a mydriatic drug (e.g. tropicamide or the like).

The amount of these ingredients in the ophthalmic composition may be appropriately selected depending on the types of preparations and the types of drugs, and the amounts of the respective ingredients are known in this field of technology. For instance, the amount can be appropriately chosen from a range of 0.0001 to 30 W/V %, preferably from 0.001 to 10 W/V %, relative to the total amount of preparation. More particularly, the amount of the respective ingredients relative to the total amount of the ophthalmic composition are just as follows.

With a decongestant, the amount is, for example, at 0.0001 to 0.5 W/V %, preferably at 0.0005 to 0.3 W/V % and more preferably at 0.001 to 0.1 W/V %.

With an antiphlogistic/astringent agent, the amount is, for example, at 0.0001 to 10 W/V %, preferably at 0.0001 to 5 W/V %.

With an antihistamine agent, its amount is, for example, at 0.0001 to 10 W/V %, preferably at 0.001 to 5 W/V %.

With a water-soluble vitamin, its amount is, for example, at 0.0001 to 1 W/V %, preferably at 0.0001 to 0.5 W/V %.

With an amino acid, the amount is, for example, at 0.0001 to 10 W/V %, preferably at 0.001 to 3 W/V %.

With a sulfur drug or bactericide, the amount is, for example, at 0.00001 to 10 W/V %, preferably at 0.0001 to 10 W/V %.

With a regional anesthetic, the amount is, for example, at 0.001 to 1 W/V %, preferably at 0.01 to 1 W/V %.

The ophthalmic composition of the invention may be used as it is in liquid form, or may be prepared as a suspension, a gelling agent or the like. The type of usage particularly includes eye drops (e.g. an ordinary eye drop, an eye drop for contact lenses and the like), eye washes (e.g. an ordinary eye wash, an eye wash used after removal of contact lenses and the like), solutions used upon wearing of contact lenses, solutions used when removing contact lenses and the like.

Contact lens users are liable to damage the cornea/conjunctiva such as by the drying of eyes ascribed to the use of contact lenses and develop, in most cases, dry eye symptoms. In this regard, vitamin A formulated in the ophthalmic composition of the invention has an amelioration effect on dry eye condition, for which when contact lens users make use of the ophthalmic composition of the invention, an amelioration effect on dry eye condition can be expected. Therefore, the ophthalmic composition of the invention is preferably used for contact lenses. Since an amount of an antiseptic is limited, use particularly for soft contact lenses is preferred.

The ophthalmic composition of the invention has an excellent effect on the corneal/conjunctival damage treatment and can be used as a dry eye remedy. The dry eye remedy of the invention shows a better effect when it is instilled into the eyes in an amount of 30 to 60 μl per time and three to six times per day.

The ophthalmic composition of the invention is in liquid form and when used as an eye drop, its viscosity is preferably at 1 to 50 mPa-second, more preferably at 1 to 30 mPa-second, much more preferably at 1 to 20 mPa-second and most preferably at 1 to 5 mPa-second. It will be noted that the viscosity is measured at 20° C. by use of an E-type viscometer (VISCONIC ELD-R, made by Tokyo Keiki Inc.).

The method for preparation of the ophthalmic composition of the invention is not particularly limited. For instance, the composition can be obtained by solubilizing vitamin A in purified water with the aid of polyoxyethylene polyoxypropylene glycol, followed by adding other formulation ingredients and adjusting a pH thereof. Thereafter, the composition can be aseptically filled in an appropriate container, for example, a polyethylene terephthalate container.

The invention provides a method for preventing cloudiness/precipitation as will be caused by freeze and thaw, which method comprising formulating, in an ophthalmic composition including (A) vitamin A and (B) polyoxyethylene polyoxypropylene, one or two or more selected from the group consisting of (C) trometamol, (D) a polyhydric alcohol, (E) a sugar, (F) phosphoric acid and a salt thereof, and (G) a monovalent neutral salt. In this cloudiness/precipitation preventing method, the ingredients and amounts thereof are same as those described hereinabove.

### EXAMPLES

Examples, Comparative Examples and Experimental Examples are shown to particularly illustrate the invention, which should not be construed as limited to the following Examples.

### Examples 1 to 48, Comparative Examples 1 to 3

Ophthalmic compositions (dye drops) having formulations indicated in Tables 1 to 11 were prepared and evaluated in the following way. The results are also indicated in the tables.

<Appearance Stability (Appearance Observation after Freeze And Thaw>

The respective ophthalmic compositions (eye drops) were filled (N=3) in a polyethylene terephthalate eye-drop container and a cycle of freeze (−25° C.)/thaw (25° C.) was repeated five times, followed by evaluation based on the following standards.

### Evaluation Standards

5: In the fifth cycle, the solution was clear, with no development of cloudiness/precipitation.
4: In the fourth cycle, the solution was clear with no development of cloudiness/precipitation, but with cloudiness or precipitation developed in the fifth cycle.
3: In the third cycle, the solution was clear with no development of cloudiness/precipitation, but with cloudiness or precipitation developed in the fourth cycle.
2: In the second cycle, the solution was clear with no development of cloudiness/precipitation, but with cloudiness or precipitation developed in the third cycle.
1: In the first step, cloudiness or precipitation developed.

The “clear” means “to be transparent without turbidity.”

### <VA Stability (Residual Rate (%) of Retinol Palmitate)>

The content of retinol palmitate in an ophthalmic composition was measured immediately after preparation and after storage for six months at 40° C. and 75% (severe test). The measurement was carried out by use of a liquid chromatographic method. The residual rate (%) of the retinol palmitate was calculated from the resulting content of the retinol palmitate according to the following equation.

Residual rate (%) of retinol palmitate=[content of retinol palmitate after storage/content of retinol palmitate immediately after preparation]×100

### <Evaluation>

⊚: Not less than 70%

◯: Not less than 65% to less than 70%

Δ: Not less than 60% to less than 65%

X: Less than 60%

### <Corneal/Conjunctival Damage Treatment Effect>

Rabbits were subjected to heptanol treatment (by dropping 200 μl per eye of a mixed solution of heptanol/ethanol=8:2 (by volume)) to provide a model suffered from a disorder at the corneal and conjunctival cuticle of the rabbit. Thereafter, the respective samples were continuously instilled into the eyes over 11 days (six times (100 μl/time)/day). During the course of the instillation in the eyes, fluorescein staining was carried out periodically (by dropping 50 μl per eye of 2% fluorescein), under which the corneal and conjunctival damage treatment effect was assessed based on the Lenp judgment standards on a fifteen point-scale (a score immediately after the heptanol treatment was set at 15 points and decreased according to the degree of improvement). The results of the assessment at the fifth day are shown in Tables 1 to 11.

TABLE 1

| | Formulation | Example | | | | | |
|---|---|---|---|---|---|---|---|
| Ingredient | (W/V %) | 1 | 2 | 3 | 4 | 5 | 6 |
| (A) | Retinol palmitate | 50,000 | 50,000 | 50,000 | 50,000 | 50,000 | 50,000 |
| | | units | units | units | units | units | units |
| (B) | Polyoxyethylene (200) | 1 | 1 | 1 | 1 | 1 | 1 |
| | polyoxypropylene (70) | | | | | | |
| | glycol *1 | | | | | | |
| (C) | Trometamol | — | — | 1 | — | — | — |
| (D) | Glycerine | 0.001 | 0.05 | — | — | — | — |
| (E) | Trehalose | — | — | — | 0.5 | — | — |
| (F) | Sodium dihydrogen | — | — | — | — | 0.5 | — |
| | phosphate | | | | | | |
| (G) | Sodium chloride | — | — | — | — | — | 0.5 |
| Buffering | Boric acid | 1 | 1 | 1 | 1 | 1 | 1 |
| agent | | | | | | | |
| Stabilizing | Sodium edetate | 0.1 | 0.1 | 0.1 | 0.1 | 0.1 | 0.1 |
| agent | | | | | | | |
| Others | d-α-Tocopherol | 0.05 | 0.05 | 0.05 | 0.05 | 0.05 | 0.05 |
| | acetate | | | | | | |
| | Dibutylhydroxy- | 0.005 | 0.005 | 0.005 | 0.005 | 0.005 | 0.005 |
| | toluene | | | | | | |

| | | | | | | |
|---|---|---|---|---|---|---|
| Diluted hydrochloric | Appropriate | Appropriate | Appropriate | Appropriate | Appropriate | Appropriate |
| acid/sodium hydroxide | amount | amount | amount | amount | amount | amount |
| (pH = 7) | | | | | | |
| Purified water | Balance | Balance | Balance | Balance | Balance | Balance |

| | | | | | | | | | | | | |
|---|---|---|---|---|---|---|---|---|---|---|---|---|
| Total | 100 | ml | 100 | ml | 100 | ml | 100 | ml | 100 | ml | 100 | ml |

| | | | | | | |
|---|---|---|---|---|---|---|
| Appearance stability | 2 | 4 | 4 | 4 | 4 | 4 |
| Evaluation of VA stability | ◯ | ◯ | ⊚ | ◯ | ◯ | ◯ |
| Corneal/conjunctival damage | 9 | 9 | 9 | 9 | 9 | 9 |
| treatment effect | | | | | | |

*1: Uniloob 70DP-950B, JPE (made by NOF Corporation) or Lutrol F127, JPE (made by BASF)

TABLE 2

| | Formulation | Example | | | | | | |
|---|---|---|---|---|---|---|---|---|
| Ingredient | (W/V %) | 7 | 8 | 9 | 10 | 11 | 12 | 13 |
| (A) | Retinol palmitate | 50,000 | 50,000 | 50,000 | 50,000 | 50,000 | 50,000 | 50,000 |
| | | units | units | units | units | units | units | units |
| (B) | Polyoxyethylene (200) | 0.4 | 2 | 5 | 1 | 1 | 1 | 1 |
| | polyoxypropylene (70) | | | | | | | |
| | glycol *1 | | | | | | | |
| (C) | Trometamol | — | — | — | — | 0.001 | 0.01 | 0.1 |
| (D) | Glycerine | 0.05 | 0.05 | 0.05 | 0.005 | — | — | — |
| (E) | Trehalose | — | — | — | — | — | — | — |
| (F) | Sodium dihydrogen | — | — | — | — | — | — | — |
| | phosphate | | | | | | | |
| (G) | Sodium chloride | — | — | — | — | — | — | — |
| Buffering | Boric acid | 1 | 1 | 1 | 1 | 1 | 1 | 1 |
| agent | | | | | | | | |
| Stabilizing | Sodium edetate | 0.1 | 0.1 | 0.1 | 0.1 | 0.1 | 0.1 | 0.1 |
| agent | | | | | | | | |
| Others | d-α-Tocopherol | 0.05 | 0.05 | 0.05 | 0.05 | 0.05 | 0.05 | 0.05 |
| | acetate | | | | | | | |
| | Dibutylhydroxy- | 0.005 | 0.005 | 0.005 | 0.005 | 0.005 | 0.005 | 0.005 |
| | toluene | | | | | | | |

| | | | | | | | |
|---|---|---|---|---|---|---|---|
| Diluted hydrochloric | Appropriate | Appropriate | Appropriate | Appropriate | Appropriate | Appropriate | Appropriate |
| acid/sodium hydroxide | amount | amount | amount | amount | amount | amount | amount |
| (pH = 7) | | | | | | | |
| Purified water | Balance | Balance | Balance | Balance | Balance | Balance | Balance |

| | | | | | | | | | | | | | | |
|---|---|---|---|---|---|---|---|---|---|---|---|---|---|---|
| Total | 100 | ml | 100 | ml | 100 | ml | 100 | ml | 100 | ml | 100 | ml | 100 | ml |

| | | | | | | | |
|---|---|---|---|---|---|---|---|
| Appearance stability | 4 | 4 | 4 | 3 | 2 | 3 | 4 |
| Evaluation of VA stability | ◯ | ◯ | ◯ | ◯ | ◯ | ⊚ | ⊚ |
| Corneal/conjunctival damage | 9 | 9 | 9 | 9 | 9 | 9 | 9 |
| treatment effect | | | | | | | |

*1: Uniloob 70DP-950B, JPE (made by NOF Corporation) or Lutrol F127, JPE (made by BASF)

TABLE 3

| | Formulation | Example | | | |
|---|---|---|---|---|---|
| Ingredient | (W/V %) | 14 | 15 | 16 | 17 |
| (A) | Retinol palmitate | 50,000 | 50,000 | 50,000 | 50,000 |
| | | units | units | units | units |
| (B) | Polyoxyethylene (200) | 1.0 | 1.0 | 1.0 | 1.0 |
| | polyoxypropylene (70) | | | | |
| | glycol *1 | | | | |
| (C) | Trometamol | 2 | — | — | — |
| (D) | Glycerine | — | — | — | — |
| (E) | Trehalose | — | 0.01 | — | — |
| (F) | Sodium dihydrogen | — | — | 0.01 | — |
| | phosphate | | | | |
| (G) | Sodium chloride | — | — | — | 0.01 |
| Buffering | Boric acid | — | 1 | 1 | 1 |
| agent | | | | | |
| Stabilizing | Sodium edetate | — | 0.1 | 0.1 | 0.1 |
| agent | | | | | |
| Others | d-α-Tocopherol acetate | 0.05 | 0.05 | 0.05 | 0.05 |
| | Dibutylhydroxytoluene | 0.005 | 0.005 | 0.005 | 0.005 |
| | Diluted hydrochloric acid/ | Appropriate | Appropriate | Appropriate | Appropriate |
| | sodium hydroxide (pH = 7) | amount | amount | amount | amount |
| | Purified water | Balance | Balance | Balance | Balance |

| | | | | |
|---|---|---|---|---|
| Total | 100 ml | 100 ml | 100 ml | 100 ml |
| Appearance stability | 4 | 3 | 3 | 3 |
| Evaluation of VA stability | ⊚ | ◯ | ◯ | ◯ |
| Corneal/conjunctival damage treatment | 9 | 9 | 9 | 9 |
| effect | | | | |

*1: Uniloob 70DP-950B, JPE (made by NOF Corporation) or Lutrol F127, JPE (made by BASF)

TABLE 4

| | Formulation | Comparative Example | | | Example | | |
|---|---|---|---|---|---|---|---|
| Ingredient | (W/V %) | 1 | 2 | 3 | 18 | 19 | 20 |
| (A) | Retinol palmitate | 50,000 | 50,000 | 50,000 | 50,000 | 50,000 | 50,000 |
| | | units | units | units | units | units | units |
| (B) | Polyoxyethylene (200) | 1 | 1 | 1 | 1 | 1 | 1 |
| | polyoxypropylene (70) | | | | | | |
| | glycol *1 | | | | | | |
| (C) | Trometamol | — | — | — | 0.5 | 0.5 | — |
| (D) | Glycerine | — | — | — | 0.5 | — | 0.5 |
| | Propylene glycol | — | — | — | — | 0.5 | 0.5 |
| (E) | Trehalose | — | — | — | — | — | — |
| (F) | Sodium dihydrogen | — | — | — | — | — | — |
| | phosphate | | | | | | |
| (G) | Sodium chloride | — | — | — | — | — | — |
| Buffering | Boric acid | — | 1 | — | 1 | 1 | 1 |
| agent | | | | | | | |
| Stabilizing | Sodium edetate | — | — | 0.1 | 0.1 | 0.1 | 0.1 |
| agent | | | | | | | |
| Others | d-α-Tocopherol | 0.05 | 0.05 | 0.05 | 0.05 | 0.05 | 0.05 |
| | acetate | | | | | | |
| | Dibutylhydroxy- | 0.005 | 0.005 | 0.005 | 0.005 | 0.005 | 0.005 |
| | toluene | | | | | | |

| | | | | | | |
|---|---|---|---|---|---|---|
| Diluted hydrochloric | Appropriate | Appropriate | Appropriate | Appropriate | Appropriate | Appropriate |
| acid/sodium hydroxide | amount | amount | amount | amount | amount | amount |
| (pH = 7) | | | | | | |
| Purified water | Balance | Balance | Balance | Balance | Balance | Balance |

| | | | | | | | | | | | | |
|---|---|---|---|---|---|---|---|---|---|---|---|---|
| Total | 100 | ml | 100 | ml | 100 | ml | 100 | ml | 100 | ml | 100 | ml |

| | | | | | | |
|---|---|---|---|---|---|---|
| Appearance stability | 1 | 1 | 1 | 5 | 5 | 4 |
| Evaluation of VA stability | ◯ | ◯ | ◯ | ⊚ | ⊚ | ◯ |
| Corneal/conjunctival damage | 9 | 9 | 9 | 9 | 9 | 9 |
| treatment effect | | | | | | |

*1: Uniloob 70DP-950B, JPE (made by NOF Corporation) or Lutrol F127, JPE (made by BASF)

TABLE 5

| | Formulation | Example | | | |
|---|---|---|---|---|---|
| Ingredient | (W/V %) | 21 | 22 | 23 | 24 |
| (A) | Retinol palmitate | 50,000 | 50,000 | 50,000 | 50,000 |
| | | units | units | units | units |
| (B) | Polyoxyethylene (200) | 1 | 1 | 1 | 1 |
| | polyoxypropylene (70) | | | | |
| | glycol *1 | | | | |
| (C) | Trometamol | 2 | — | 0.03 | 1 |
| (D) | Glycerine | 0.02 | 1 | — | 0.6 |
| (E) | Trehalose | — | 1 | — | — |
| (F) | Sodium dihydrogen | — | — | 0.5 | — |
| | phosphate | | | | |
| (G) | Sodium chloride | — | — | 0.5 | 0.3 |
| Others | Sodium edetate | 0.1 | 0.1 | 0.1 | 0.1 |
| | d-α-Tocopherol acetate | 0.05 | 0.05 | 0.05 | 0.05 |
| | Dibutylhydroxytoluene | 0.005 | 0.005 | 0.005 | 0.005 |
| | Diluted hydrochloric acid/ | Appropriate | Appropriate | Appropriate | Appropriate |
| | sodium hydroxide (pH = 7) | amount | amount | amount | amount |
| | Purified water | Balance | Balance | Balance | Balance |

| | | | | |
|---|---|---|---|---|
| Total | 100 ml | 100 ml | 100 ml | 100 ml |
| Appearance stability | 5 | 4 | 5 | 5 |
| Evaluation of VA stability | ⊚ | ◯ | ⊚ | ⊚ |
| Corneal/conjunctival damage treatment | 9 | 9 | 9 | 9 |
| effect | | | | |

*1: Uniloob 70DP-950B, JPE (made by NOF Corporation) or Lutrol F127, JPE (made by BASF)

TABLE 6

| | Formulation | Example | | | |
|---|---|---|---|---|---|
| Ingredient | (W/V %) | 25 | 26 | 27 | 28 |
| (A) | Retinol palmitate | 50,000 | 50,000 | 50,000 | 50,000 |
| | | units | units | units | units |
| (B) | Polyoxyethylene (200) | 1 | 1 | 1 | 1 |
| | polyoxypropylene (70) | | | | |
| | glycol *1 | | | | |
| (C) | Trometamol | 2 | 0.5 | 1 | 0.6 |
| (D) | Glycerine | — | 0.2 | 1 | 0.3 |
| (E) | Trehalose | 1 | 0.2 | — | 0.1 |
| (F) | Sodium dihydrogen | 1 | — | 0.5 | — |
| | phosphate | | | | |
| (G) | Sodium chloride | — | — | — | 0.1 |
| Others | Sodium edetate | 0.1 | 0.1 | 0.1 | 0.1 |
| | d-α-Tocopherol acetate | 0.05 | 0.05 | 0.05 | 0.05 |
| | Dibutylhydroxytoluene | 0.005 | 0.005 | 0.005 | 0.005 |
| | Diluted hydrochloric acid/ | Appropriate | Appropriate | Appropriate | Appropriate |
| | sodium hydroxide (pH = 7) | amount | amount | amount | amount |
| | Purified water | Balance | Balance | Balance | Balance |

| | | | | |
|---|---|---|---|---|
| Total | 100 ml | 100 ml | 100 ml | 100 ml |
| Appearance stability | 5 | 5 | 5 | 5 |
| Evaluation of VA stability | ⊚ | ⊚ | ⊚ | ⊚ |
| Corneal/conjunctival damage treatment | 9 | 9 | 9 | 9 |
| effect | | | | |

*1: Uniloob 70DP-950B, JPE (made by NOF Corporation) or Lutrol F127, JPE (made by BASF)

TABLE 7

| | Formulation | Example | | | |
|---|---|---|---|---|---|
| Ingredient | (W/V %) | 29 | 30 | 31 | 32 |
| (A) | Retinol palmitate | 50,000 | 70,000 | 70,000 | 100,000 |
| | | units | units | units | units |
| (B) | Polyoxyethylene (200) | 0.5 | 0.5 | 2 | 2 |
| | polyoxypropylene (70) | | | | |
| | glycol *1 | | | | |
| (C) | Trometamol | 0.2 | 1 | 0.5 | 1 |
| (D) | Glycerine | 1 | 0.3 | 0.2 | 0.5 |
| (E) | Trehalose | — | 0.5 | 1 | — |
| (F) | Sodium dihydrogen | — | — | 0.2 | 1.5 |
| | phosphate | | | | |
| (G) | Sodium chloride | — | — | — | 0.3 |
| Others | Castor oil | 0.05 | 0.1 | 0.5 | 0.2 |
| | Polyoxyethylene | 0.3 | 0.1 | — | — |
| | hardened castor oil 60 | | | | |
| | Sodium hyaluronate | 0.02 | — | — | — |
| | Polyvinylpyrrolidone | — | — | 0.1 | — |
| | Sodium chondroitin | — | 0.1 | — | 0.1 |
| | sulfate | | | | |
| | Potassium L-aspartate | — | 1 | — | 1 |
| | Boric acid | 1.5 | 0.5 | 0.5 | 0.5 |
| | Borax | — | — | — | 0.2 |
| | l-Menthol | — | — | 0.005 | — |
| | dl-Camphor | — | — | 0.002 | — |
| | d-Borneol | — | — | 0.003 | — |
| | Potassium sorbate | — | — | 0.1 | 0.1 |
| | Sodium edetate | 0.1 | 0.1 | 0.1 | 0.1 |
| | d-α-Tocopherol acetate | 0.05 | 0.05 | 0.05 | 0.05 |
| | Dibutylhydroxytoluene | 0.005 | 0.005 | 0.005 | 0.005 |
| | Diluted hydrochloric acid/ | Appropriate | Appropriate | Appropriate | Appropriate |
| | soditm hydroxide (pH = 7) | amount | amount | amount | amount |
| | Purified water | Balance | Balance | Balance | Balance |

| | | | | |
|---|---|---|---|---|
| Total | 100 ml | 100 ml | 100 ml | 100 ml |
| Appearance stability | 5 | 5 | 5 | 5 |
| Evaluation of VA stability | ⊚ | ◯ | ◯ | ◯ |
| Corneal/conjunctival damage treatment | 9 | 8 | 8 | 7 |
| effect | | | | |

*1: Uniloob 70DP-950B, JPE (made by NOF Corporation) or Lutrol F127, JPE (made by BASF)

TABLE 8

| | Formulation | Example | |
|---|---|---|---|
| Ingredient | (W/V %) | 33 | 34 |
| (A) | Retinol palmitate | 100,000 | 200,000 |
| | | units | units |
| (B) | Polyoxyethylene (200) | 3 | 5 |
| | polyoxypropylene (70) glycol *1 | | |
| (C) | Trometamol | 0.5 | 1 |
| (D) | Glycerine | — | 1 |
| (E) | Trehalose | 1 | — |
| (F) | Sodium dihydrogen phosphate | — | — |
| (G) | Sodium chloride | — | 0.2 |
| Others | Castor oil | — | — |
| | Polyoxyethylene hardened castor | 0.2 | — |
| | oil 60 | | |
| | Sodium hyaluronate | 0.02 | — |
| | Polyvinylpyrrolidone | — | — |
| | Sodium chondroitin sulfate | — | 0.1 |
| | Potassium L-aspartate | — | 1 |
| | Boric acid | 0.5 | 0.5 |
| | Borax | — | 0.2 |
| | l-Menthol | — | — |
| | dl-Camphor | — | — |
| | d-Borneol | — | — |
| | Potassium sorbate | — | — |
| | Sodium edetate | 0.1 | 0.1 |
| | d-α-Tocopherol acetate | 0.05 | 0.05 |
| | Dibutylhydroxytoluene | 0.005 | 0.005 |
| | Diluted hydrochloric acid/ | Appropriate | Appropriate |
| | sodium hydroxide (pH = 7) | amount | amount |
| | Purified water | Balance | Balance |

| | | |
|---|---|---|
| Total | 100 ml | 100 ml |
| Appearance stability | 5 | 5 |
| Evaluation of VA stability | ◯ | ◯ |
| Corneal/conjunctival damage treatment effect | 7 | 6 |

*1: Uniloob 70DP-950B, JPE (made by NOF Corporation) or Lutrol F127, JPE (made by BASF)

TABLE 9

| | Formulation | Example | | | |
|---|---|---|---|---|---|
| Ingredient | (W/V %) | 35 | 36 | 37 | 38 |
| (A) | Retinol palmitate | 50,000 | 70,000 | 70,000 | 100,000 |
| | | units | units | units | units |
| (B) | Polyoxyethylene (200) | 0.5 | 0.5 | 1 | 1 |
| | polyoxypropylene (70) glycol *1 | | | | |
| (C) | Trometamol | 1 | — | 0.1 | — |
| (D) | Glycerine | 2 | 1 | — | — |
| (E) | Trehalose | — | — | — | 0.2 |
| | Xylitol | 0.2 | — | — | — |
| | Sorbitol | — | 0.5 | — | — |
| | Mannitol | — | — | 1 | — |
| (F) | Sodium dihydrogen phosphate | — | 0.5 | — | 1 |
| (G) | Sodium chloride | — | 0.3 | 0.2 | 0.6 |
| Others | Polyoxyethylene hardened | — | 0.1 | — | — |
| | castor oil 60 | | | | |
| | Polysorbate 80 | 0.2 | — | 0.05 | — |
| | Tetrahydrozoline hydrochloride | 0.05 | — | 0.05 | — |
| | Neostigmine methylsulfate | 0.005 | — | 0.005 | — |
| | Chlorpheniramine maleate | 0.03 | 0.03 | 0.03 | 0.03 |
| | Pyridoxine hydrochloride | — | 0.05 | 0.05 | 0.05 |
| | Dipotassium glycyrrhizinate | — | — | — | 0.25 |
| | Boric acid | 0.5 | 0.5 | 2 | 0.5 |
| | Borax | — | 0.2 | — | 0.5 |
| | l-Menthol | 0.005 | — | — | 0.005 |
| | dl-Camphor | 0.002 | — | — | 0.002 |
| | d-Borneol | 0.003 | — | — | 0.003 |
| | Potassium sorbate | — | — | — | 0.1 |
| | Sodium edetate | 0.1 | 0.1 | 0.1 | 0.1 |
| | d-a-Tocopherol acetate | 0.05 | 0.05 | 0.05 | 0.05 |
| | Dibutylhydroxytoluene | 0.005 | 0.005 | 0.005 | 0.005 |
| | Diluted hydrochloric acid/ | Appropriate | Appropriate | Appropriate | Appropriate |
| | sodium hydroxide (pH = 7) | amount | amount | amount | amount |
| | Purified water | Balance | Balance | Balance | Balance |

| | | | | |
|---|---|---|---|---|
| Total | 100 ml | 100 ml | 100 ml | 100 ml |
| Appearance stability | 5 | 4 | 5 | 4 |
| Evaluation of VA stability | ⊚ | Δ | ◯ | Δ |
| Corneal/conjunctival damage treatment effect | 10 | 8 | 8 | 7 |

*1: Uniloob 70DP-950B, JPE (made by NOF Corporation) or Lutrol F127, JPE (made by BASF)

TABLE 10

| | Formulation | Example | | |
|---|---|---|---|---|
| Ingredient | (W/V %) | 39 | 40 | 41 |
| (A) | Retinol palmitate | 100,000 | 150,000 | 200,000 |
| | | units | units | units |
| (B) | Polyoxyethylene (200) | 2 | 4 | 5 |
| | polyoxypropylene (70) | | | |
| | glycol *1 | | | |
| (C) | Trometamol | 1 | — | 1 |
| (D) | Glycerine | — | 1.5 | — |
| (E) | Trehalose | — | — | 0.5 |
| | Xylitol | — | — | — |
| | Sorbitol | 0.2 | — | — |
| | Mannitol | — | 0.3 | — |
| (F) | Sodium dihydrogen phosphate | 1 | 1 | — |
| (G) | Sodium chloride | 0.3 | — | 0.3 |
| Others | Polyoxyethylene hardened | 0.4 | — | — |
| | castor oil 60 | | | |
| | Polysorbate 80 | — | — | 0.05 |
| | Tetrahydrozoline hydrochloride | 0.05 | — | 0.05 |
| | Neostigmine methylsulfate | 0.005 | — | 0.005 |
| | Chlorpheniramine maleate | 0.03 | 0.03 | 0.03 |
| | Pyridoxine hydrochloride | 0.05 | 0.05 | 0.05 |
| | Dipotassium glycyrrhizinate | — | 0.25 | — |
| | Boric acid | 1 | 0.5 | — |
| | Borax | — | 0.1 | — |
| | l-Menthol | — | 0.005 | 0.2 |
| | dl-Camphor | — | 0.002 | — |
| | d-Borneol | — | 0.003 | — |
| | Potassium sorbate | — | — | 0.1 |
| | Sodium edetate | 0.1 | 0.1 | 0.1 |
| | d-α-Tocopherol acetate | 0.05 | 0.05 | 0.05 |
| | Dibutylhydroxytoluene | 0.005 | 0.005 | 0.005 |
| | Diluted hydrochloric acid/ | Appropriate | Appropriate | Appropriate |
| | sodium hydroxide (pH = 7) | amount | amount | mount |
| | Purified water | Balance | Balance | Balance |

| | | | |
|---|---|---|---|
| Total | 100 ml | 100 ml | 100 ml |
| Appearance stability | 5 | 4 | 5 |
| Evaluation of VA stability | ◯ | Δ | ◯ |
| Corneal/conjunctival damage treatment effect | 7 | 7 | 6 |

*1: Uniloob 70DP-950B, JPE (made by NOF Corporation) or Lutrol F127, JPE (made by BASF)

TABLE 11

| | Formulation | Example | | | | | | |
|---|---|---|---|---|---|---|---|---|
| Ingredient | (W/V %) | 42 | 43 | 44 | 45 | 46 | 47 | 48 |
| (A) | Retinol palmitate | 150,000 | 150,000 | 150,000 | 150,000 | 150,000 | 300,000 | 500,000 |
| | | units | units | units | units | units | units | units |
| (B) | Polyoxyethylene (200) | 5 | 3 | 3 | 3 | 3 | 5 | 5 |
| | polyoxypropylene (70) | | | | | | | |
| | glycol *1 | | | | | | | |
| (C) | Trometamol | 1 | — | — | — | — | 5 | 5 |
| (D) | Glycerine | — | 0.5 | 0.5 | 0.5 | — | — | — |
| (E) | Trehalose | — | 0.25 | — | — | 0.25 | 0.5 | — |
| (F) | Sodium dihydrogen | — | — | 0.25 | — | 0.25 | — | 0.5 |
| | phosphate | | | | | | | |
| (G) | Sodium chloride | — | — | — | 0.3 | — | 0.5 | 0.5 |
| Buffering | Boric acid | 1 | 1 | 1 | 1 | 1 | 0.1 | 0.1 |
| agent | | | | | | | | |
| Stabilizing | Sodium edetate | 0.1 | 0.1 | 0.1 | 0.1 | 0.1 | 0.1 | 0.1 |
| agent | | | | | | | | |
| Others | d-α-Tocopherol | 0.05 | 0.05 | 0.05 | 0.05 | 0.05 | 0.05 | 0.05 |
| | acetate | | | | | | | |
| | Dibutylhydroxy- | 0.005 | 0.005 | 0.005 | 0.005 | 0.005 | 0.005 | 0.005 |
| | toluene | | | | | | | |

| | | | | | | | |
|---|---|---|---|---|---|---|---|
| Diluted hydrochloric | Appropriate | Appropriate | Appropriate | Appropriate | Appropriate | Appropriate | Appropriate |
| acid/sodium hydroxide | amount | amount | amount | amount | amount | amount | amount |
| (pH = 7) | | | | | | | |
| Purified water | Balance | Balance | Balance | Balance | Balance | Balance | Balance |

| | | | | | | | | | | | | | | |
|---|---|---|---|---|---|---|---|---|---|---|---|---|---|---|
| Total | 100 | ml | 100 | ml | 100 | ml | 100 | ml | 100 | ml | 100 | ml | 100 | ml |

| | | | | | | | |
|---|---|---|---|---|---|---|---|
| Appearance stability | 4 | 4 | 4 | 4 | 4 | 5 | 5 |
| Evaluation of VA stability | ◯ | Δ | Δ | Δ | Δ | ◯ | ◯ |
| Corneal/conjunctival damage | 7 | 7 | 7 | 7 | 7 | 5 | 5 |
| treatment effect | | | | | | | |

*1: Uniloob 70DP-950B, JPE (made by NOF Corporation) or Lutrol F127, JPE (made by BASF)

### Experimental Examples 1 to 12

The ophthalmic compositions (eye-drops) having the formulations indicated in Tables 12, 13 were obtained in such way that vitamin A, polyoxyethylene polyoxypropylene glycol and an antioxidant were preliminarily dissolved at 85° C., and the preliminarily dissolved matter was solubilized in sterilized, purified water and cooled, to which water-soluble ingredients such as trometamol and the like were added, followed adjusting the pH (20° C.). 15 ml of the resulting ophthalmic composition was filled in a 15 ml filter-attached eye-drop container (made of polyethylene terephthalate). It will be noted that the ophthalmic compositions of Experimental Examples 1 to 12 had satisfactory antiseptic power. The ophthalmic compositions were subjected to evaluation of the corneal/conjunctival damage treatment effect. The results are also shown in the tables.

TABLE 12

| Formulation | | Experimental Example | | | | | |
|---|---|---|---|---|---|---|---|
| (W/V %) | | 1 | 2 | 3 | 4 | 5 | 6 |
| (A) | Retinol palmitate | 50,000 | 100,000 | 150,000 | 200,000 | 300,000 | 500,000 |
| | | units | units | units | units | units | units |
| (B) | Polyoxyethylene (200) | 2 | 2 | 3 | 4 | 5 | 5 |
| | polyoxypropylene (70) | | | | | | |
| | glycol *1 | | | | | | |
| | Polyoxyethylene (160) | — | — | — | — | — | — |
| | polyoxypropylene (30) | | | | | | |
| | glycol *2 | | | | | | |
| | Polyoxyethylene (54) | — | — | — | — | — | — |
| | polyoxypropylene (39) | | | | | | |
| | glycol *3 | | | | | | |
| (F) | Sodium chloride | 0.9 | 0.9 | 0.9 | 0.9 | 0.9 | 0.9 |
| Others | Boric acid | 1 | 1 | 1 | 1 | 1 | 1 |
| | Borax | 0.1 | 0.1 | 0.1 | 0.1 | 0.1 | 0.1 |
| | Sodium edetate | 0.1 | 0.1 | 0.1 | 0.1 | 0.1 | 0.1 |
| | Benzalkonium chloride | — | — | — | — | — | — |

| | | | | | | |
|---|---|---|---|---|---|---|
| Diluted hydrochloric | Appropriate | Appropriate | Appropriate | Appropriate | Appropriate | Appropriate |
| acid/sodium hydroxide | amount | amount | amount | amount | amount | amount |
| Purified water | Balance | Balance | Balance | Balance | Balance | Balance |

| | | | | | | | | | | | | |
|---|---|---|---|---|---|---|---|---|---|---|---|---|
| Total | 100 | ml | 100 | ml | 100 | ml | 100 | ml | 100 | ml | 100 | ml |

| | | | | | | |
|---|---|---|---|---|---|---|
| Corneal/conjunctival damage | 10 | 9 | 9 | 8 | 6 | 6 |
| treatment effect | | | | | | |

*1: Uniloob 70DP-950B, JPE (made by NOF Corporation) or Lutrol F127, JPE (made by BASF)
*2: Plonon #188P, JPE, made by NOF Corporation
*3: Plonon #235P, JPE, made by NOF Corporation

TABLE 13

| Formulation | | Experimental Example | | | | | |
|---|---|---|---|---|---|---|---|
| (W/V %) | | 7 | 8 | 9 | 10 | 11 | 12 |
| (A) | Retinol palmitate | 0 | 30,000 | 40,000 | 50,000 | 300,000 | 300,000 |
| | | unit | units | units | units | units | units |
| (B) | Polyoxyethylene (200) | 2 | 2 | 2 | 2 | — | — |
| | polyoxypropylene (70) | | | | | | |
| | glycol *1 | | | | | | |
| | Polyoxyethylene (160) | — | — | — | — | 5 | — |
| | polyoxypropylene (30) | | | | | | |
| | glycol *2 | | | | | | |
| | Polyoxyethylene (54) | — | — | — | — | — | 5 |
| | polyoxypropylene (39) | | | | | | |
| | glycol *3 | | | | | | |
| (F) | Sodium chloride | 0.9 | 0.9 | 0.9 | 0.9 | 0.9 | 0.9 |
| Others | Boric acid | 1 | 1 | 1 | 1 | 1 | 1 |
| | Borax | 0.1 | 0.1 | 0.1 | 0.1 | 0.1 | 0.1 |
| | Sodium edetate | 0.1 | 0.1 | 0.1 | 0.1 | 0.1 | 0.1 |
| | Benzalkonium chloride | — | — | — | 0.1 | — | — |

| | | | | | | |
|---|---|---|---|---|---|---|
| Diluted hydrochloric | Appropriate | Appropriate | Appropriate | Appropriate | Appropriate | Appropriate |
| acid/sodium hydroxide | amount | amount | amount | amount | amount | amount |
| Purified water | Balance | Balance | Balance | Balance | Balance | Balance |

| | | | | | | | | | | | | |
|---|---|---|---|---|---|---|---|---|---|---|---|---|
| Total | 100 | ml | 100 | ml | 100 | ml | 100 | ml | 100 | ml | 100 | ml |

| | | | | | | |
|---|---|---|---|---|---|---|
| Corneal/conjunctival damage | 13 | 14 | 12 | 13 | 6 | 6 |
| treatment effect | | | | | | |

*1: Uniloob 70DP-950B, JPE (made by NOF Corporation) or Lutrol F127, JPE (made by BASF)
*2: Plonon #188P, JPE, made by NOF Corporation
*3: Plonon #235P, JPE, made by NOF Corporation

1. An ophthalmic composition, characterized by comprising (A) vitamin A, (B) polyoxyethylene polyoxypropylene glycol, and one or two or more selected from the group consisting of (C) trometamol, (D) a polyhydric alcohol, (E) a sugar, (F) phosphoric acid and its salt, and (G) a monovalent neutral salt.

2. The ophthalmic composition of claim 1, wherein two or more selected from the ingredients (C) to (G) are contained.

3. The ophthalmic composition of claim 1 or 2, wherein the ingredient (D) is glycerine, the ingredient (E) is xylitol, sorbitol, mannitol or trehalose, the ingredient (F) is sodium dihydrogen phosphate, and the ingredient (G) is sodium chloride.

4. The ophthalmic composition of claim 1, wherein a total amount of the ingredients (C) to (G) is 0.001 to 5 W/V %.

5. The ophthalmic composition of claim 1, wherein an amount of the ingredient (B) is not larger than 5 W/V %.

6. The ophthalmic composition of claim 1, wherein the ingredient (A) is one or two or more selected from the group consisting of retinol palmitate, retinol acetate and retinoic acid.

7. The ophthalmic composition of claim 1, wherein an amount of the ingredient (A) is 50,000 to 500,000 units/100 ml.

8. The ophthalmic composition of claim 1, wherein an amount of a cationic surfactant and a hydrophobic antiseptic are not larger than 0.004 W/V %, respectively.

9. The ophthalmic composition of claim 1, wherein no antiseptic is formulated.

10. The ophthalmic composition of claim 1, wherein said composition is the ophthalmic composition for contact lens.

11. The ophthalmic composition of claim 7, wherein said composition is dry eye remedy.

12. A method for preventing cloudiness/precipitation in the course of freezing and thawing comprising;
formulating one or two or more selected from the group consisting of (C) trometamol, (D) a polyhydric alcohol, (E) a sugar, (F) phosphoric acid and its salt, and (G) a monovalent neutral salt in an ophthalmic composition containing (A) vitamin A and (B) polyoxyethylene polyoxypropylene glycol.
